(19) United States
(12) Patent Application Publication
(10) Pub. No.: US 2006/0247321 A1
(43) Pub. Date: Nov. 2, 2006

(54) Abnormal Cannabidiols as agents useful in combination therapy for lowering intraocular pressure

(76) Inventor: June Chen, San Juan Capistrano, CA (US)

Correspondence Address: Robert J. Baran (T2-7H);ALLERGAN, INC., Legal Department, 2525 Dupont Drive, Irvine, CA, 92612, US

(21) Appl. No.: 11/121,528

(22) Filed: May 2, 2005

(51) Int. Cl. A61K 31/05

(52) U.S. Cl. 514/734

(57) ABSTRACT

The invention relates to the use of Abnormal Cannabidiols in a combination with a drug selected from the group consisting of cholinergic agonists, chlolinesterase inhibitors, prostamides and prostaglandins and the like, or pharmaceutically acceptable salts or prodrugs thereof as potent ocular hypotensives. Said combinations are particularly suitable for the management of glaucoma. In particular said Abnormal Cannibidiols are represented by formula I

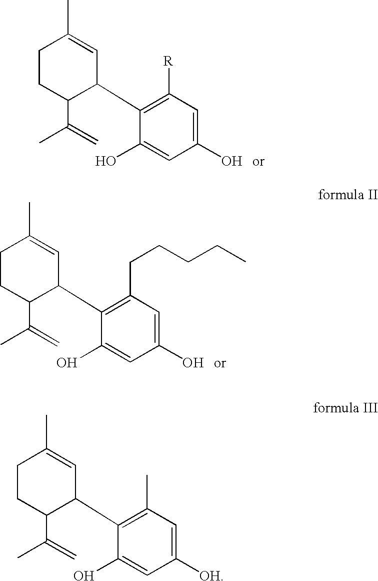

### BACKGROUND OF THE INVENTION

1. Field of the Invention

The present invention relates to the use of the combination of Abnormal Cannabidiols and agents that decrease intraocular pressure (IOP) by increasing outflow to lower the intraocular pressure of mammals and thus such combinations are useful in treating glaucoma.

2. Description of the Related Art

Ocular hypotensive agents are useful in the treatment of a number of various ocular hypertensive conditions, such as post-surgical and post-laser trabeculectomy ocular hypertensive episodes, glaucoma, and as presurgical adjuncts.

Glaucoma is a disease of the eye characterized by increased intraocular pressure. On the basis of its etiology, glaucoma has been classified as primary or secondary. For example, primary glaucoma in adults (congenital glaucoma) may be either open-angle or acute or chronic angle-closure. Secondary glaucoma results from pre-existing ocular diseases such as uveitis, intraocular tumor or an enlarged cataract.

The underlying causes of primary glaucoma are not yet known. The increased intraocular tension is due to the obstruction of aqueous humor outflow. In chronic open-angle glaucoma, the anterior chamber and its anatomic structures appear normal, but drainage of the aqueous humor is impeded. In acute or chronic angle-closure, the anterior chamber is shallow, the filtration angle is narrowed, and the iris may obstruct the trabecular meshwork at the entrance of the canal of Schlemm. Dilation of the pupil may push the root of the iris forward against the angle, and may produce pupilary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe, and may plug the drainage channel with exudates. Other common causes are intraocular tumors, enlarged cataracts, central retinal vein occlusion, trauma to the eye, operative procedures and intraocular hemorrhage.

Considering all types together, glaucoma occurs in about 2% of all persons over the age of 40 and may be asymptotic for years before progressing to rapid loss of vision. In cases where surgery is not indicated, topical α-adrenoreceptor antagonists have traditionally been the drugs of choice for treating glaucoma.

Certain Abnormal Cannabidiols are disclosed in Howlett et al, “International Union of Pharmacology. XXVII. Classification of Cannabinoid Receptors”, Pharmacological Reviews 54: 161-202, 2002.

### SUMMARY OF THE INVENTION

I have found that Abnormal Cannabidiols are potent ocular hypotensive agents which lower intraocular pressure (IOP) but do not increase outflow, i.e. uveoscleral outflow. I have further found that Abnormal Cannabidiols and homologues and derivatives thereof in combination with agents that decrease IOP by increasing aqueous outflow from the eye, are especially useful in the treatment of glaucoma.

The present invention relates to methods of treating ocular hypertension which comprises administering an effective amount of a compound represented by the formula I 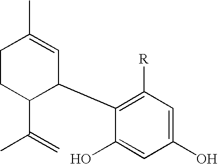
wherein R is selected from the group consisting of (CH₂)ₓ wherein x is 0 or an integer of from 1 to 7. This compound is administered in combination with an agent that decreases IOP by increasing the aqueous outflow, e.g. the uveoscleral outflow, from the eye.

In a further aspect, the present invention relates to pharmaceutical compositions comprising a therapeutically effective amount of a compound of formulae (I), in admixture with an agent that decreases IOP by increasing the aqueous outflow, e.g. the uveoscleral outflow, the resulting combination being included in a non-toxic, pharmaceutically acceptable liquid vehicle.

### DETAILED DESCRIPTION OF THE INVENTION

The present invention relates to the use of Abnormal Cannabidiols as ocular hypotensives. These therapeutic agents are represented by compounds having the formula I: 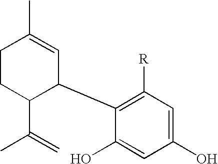
as defined above. The preferred compounds used in accordance with the present invention are encompassed by the following structural formula

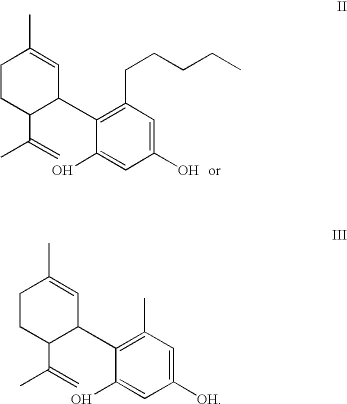

In all of the above formulae, as well as in those provided hereinafter, the straight lines represent bonds. Where there is no symbol for the atoms between the bonds, the appropriate carbon-containing radical is to be inferred. For example in formula II, the radical extending from the phenyl ring is a polymethylene (CH₂) radical terminated with a methyl radical, i.e. a butylenylmethyl radical.

This compound is administered in combination with an agent that decreases IOP by increasing the aqueous outflow, e.g. the uveoscleral outflow, from the eye.

Thus, a combination product containing an Abnormal Cannabidiols component and an ocular hypotensive agents component that enhances aqueous humor outflow via either or both the trabecular and uveoscleral outflow pathways is contemplated for effective reduction of intraocular pressure based on different modes of action and complementary pharmacology of the active ingredients.

The combination treatment with the following classes of drugs is contemplated: Cholinergic agonists including direct acting cholinergic agonists such as carbachol, pilocarpine hydrochloride, pilocarpine nitrate, pilocarpine, and the like, or pharmaceutically acceptable salts or prodrugs thereof; cholinesterase inhibitors such as demecarium, echothiophate, physostigmine, and the like, or pharmaceutically acceptable salts or prodrugs thereof; prostamides such as bimatoprost, or pharmaceutically acceptable salts or prodrugs thereof; prostaglandins including travoprost, UFO-21, cloprostenol, fluprostenol, 13,14-dihydro-cloprostenol, isopropyl unoprostone, latanoprost, prostaglandin EP analogs such as butaprost, AH-13205 and the like. These drugs act to lower IOP by increasing the aqueous outflow from the eye.

Pharmaceutical compositions may be prepared by combining a therapeutically effective amount of at least one combination of such compounds according to the present invention, as the active ingredients, with conventional ophthalmically acceptable pharmaceutical excipients, and by preparation of unit dosage forms suitable for topical ocular use. The therapeutically efficient amount typically is between about 0.0001 and about 5% (w/v), preferably about 0.001 to about 1.0% (w/v) in liquid formulations of the Abnormal Cannabinoid.

The other agent may be included in a therapeutically-efficient amount typically from about 0.0001 to about 5% (w/v), preferably from about 0.001 to about 1.0% (w/v) in the formulation.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional, pharmaceutically acceptable preservatives, stabilizers and surfactants.

Preferred preservatives that may be used in the pharmaceutical compositions of the present invention include, but are not limited to, benzalkonium chloride, chlorobutanol, thimerosal, phenylmercuric acetate and phenylmercuric nitrate. A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar vein, an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place or in conjunction with it.

The ingredients are usually used in the following amounts:

| Ingredient | Amount (% w/v) |
|---|---|
| active ingredients | about 0.001-5 |
| preservative | 0-0.10 |
| vehicle | 0-40 |
| tonicity adjustor | 1-10 |
| buffer | 0.01-10 |
| pH adjustor | q.s. pH 4.5-7.5 |
| antioxidant | as needed |
| surfactant | as needed |
| purified water | as needed to make 100% |

The actual dose of the active compounds of the present invention depends on the specific compound, and on the condition to be treated; the selection of the appropriate dose is well within the knowledge of the skilled artisan.

The ophthalmic formulations of the present invention are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution. One package may contain one or more unit doses.

Especially preservative-free solutions are often formulated in non-resealable containers containing up to about ten, preferably up to about five unit doses, where a typical unit dose is from one to about 8 drops, preferably one to about 3 drops. The volume of one drop usually is about 20-35 μl.

The present invention relates to the use of Abnormal Cannabidiols and ocular hypotensive agents that lower IOP by increasing aqueous outflow in combination and adjunctive therapies (1) to lower the IOP of mammals and thus are useful in treating glaucoma and ocular hypertension; (2) to provide neuroprotective effects to the eye of a mammal. The compositions and methods, including reduced concentrations of approved agents, have the advantage of effective treatment and/or reduction of the number and/or frequency and/or severity of unwanted side effects caused by the ocular hypotensive agents. Without limiting the invention to any particular theory or mode of operation, it is believed that the present compositions and methods take advantage of the modes of action of the Abnormal Cannabidiols component and the ocular hypotensive agents component.

A combination product containing an Abnormal Cannabidiols component and an ocular hypotensive agent component that enhances aqueous humor outflow via either or both the trabecular and uveoscleral outflow pathways is contemplated for effective reduction of intraocular pressure based on different modes of action and complementary pharmacology of the active ingredients.

The combination treatment with the following classes of drugs are contemplated: Cholinergic agonists including direct acting cholinergic agonists such as carbachol, pilocarpine hydrochloride, pilocarpine nitrate, pilocarpine, and the like, or pharmaceutically acceptable salts or prodrugs thereof; cholinesterase inhibitors such as demecarium, echothiophate, physostigmine, and the like, or pharmaceutically acceptable salts or prodrugs thereof; prostamides such as bimatoprost, or pharmaceutically acceptable salts or prodrugs thereof, prostaglandins including travoprost, UFO-21, cloprostenol, fluprostenol, 13,14-dihydro-cloprostenol, isopropyl unoprostone, latanoprost, prostaglandin EP analogs such as butaprost, AH-13205 and the like.

The invention is further illustrated by the following non-limiting Examples.

### EXAMPLE 1

### Intraocular Pressure

Intraocular pressure was measured by applanation pneumatonometry in conscious animals. The test compound was administered topically to one eye while vehicle was given to the fellow eye in a masked fashion. Ocular normotensive Beagle dogs (males, females) were dosed once daily for five days. Laser-induced unilaterally ocular hypertensive Cynomolgus monkeys (females) were dosed once daily for 4 days. Student's paired t-test was used for statistical comparisons. Differences were considered statistically significant if the P-value is less than 0.05.

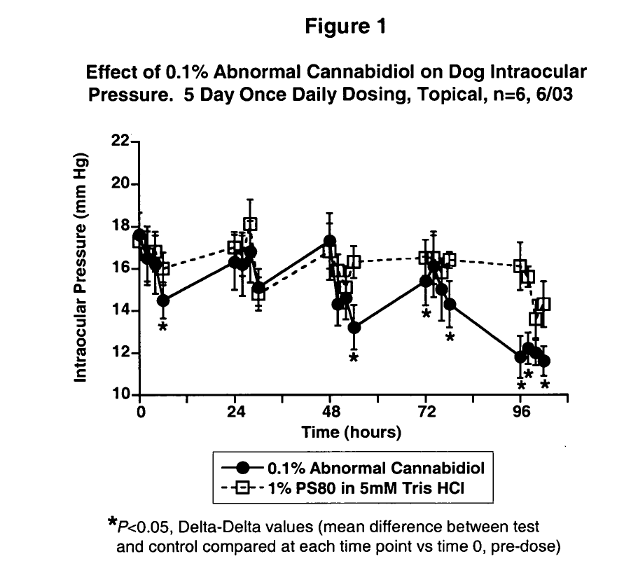

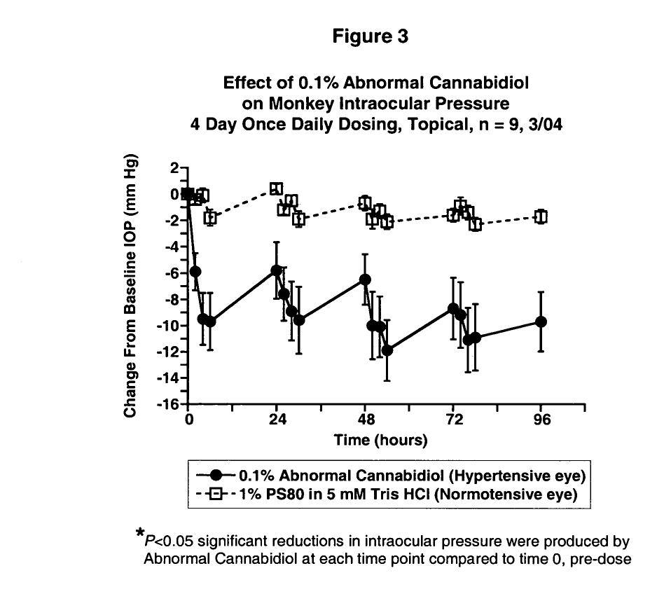

The results are shown in FIGS. 1, 2 and 3.

In particular, FIG. 1 shows the effect of 0.1% Abnormal Cannabidiol on Dog Intraocular Pressure versus time.

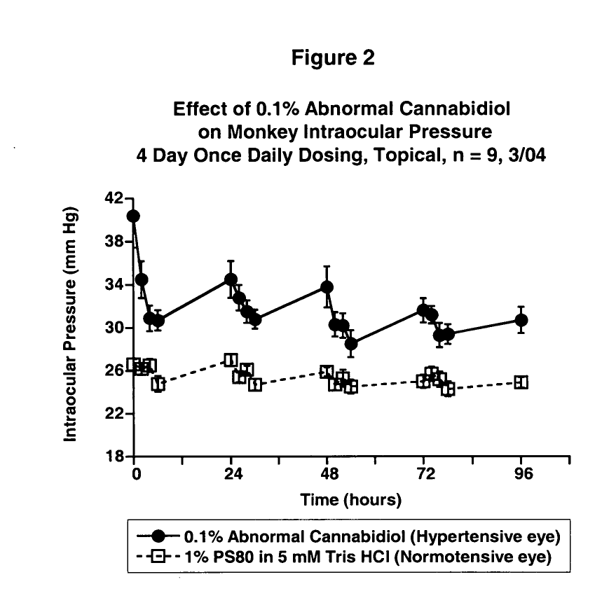

FIG. 2 shows the effect of 0.1% Abnormal Cannabidiol on Monkey Intraocular Pressure versus time.

These results show that Abnormal Cannabidiol lowers IOP.

### EXAMPLE 2

### Determination of Abnormal Cannabidiol Activity

Abnormal Cannabidiol receptor activity may be measured in accordance with the procedure disclosed in (Wagner J A et al., Hypertension 33 [part II], 429 (1999); Járai Z et al., PNAS 96, 14136 (1999), which is hereby incorporated by reference in its entirety.

### EXAMPLE 3

Measuring the Uveoscleral Outflow of Mammals (Monkeys)
Treated with Abnormal Cannabidiols
Effect of abnormal cannabidiol 0.1%, topical once-daily for 5 days, vs
vehicle on Outflow Facility (ul/min/mm Hg), mean ± sem

| | | Treatment |
|---|---|---|
| | Baseline | (abnormal cannabidiol vs |
| | (no treatment) | vehicle) |
| Treated Eye | 0.54 ± 0.10 | 0.57 ± 0.08 |
| Control Eye | 0.51 ± 0.14 | 0.65 ± 0.10 |
| Ratio, | 1.19 ± 0.17 | 0.93 ± 0.09 |
| Treated:Control | | |

Student's t-test, paired analysis, showed no significant differences
between means.
n = 6 animals

Effect of abnormal cannabidiol 0.1%, topical once-daily for 5 days, vs
vehicle on Uveosclearal Outflow (ul/min), mean ± sem

| | |
|---|---|
| Treated Eye | 0.58 ± 0.17 |
| Control Eye | 0.75 ± 0.23 |
| Ratio, Treated:Control | 0.78 ± 0.05 |

Student's t-test, paired analysis, showed no significant differences
between means.
n = 5 animals

These experimental findings indicated that abnormal cannabidiol 0.1%, given topically once-daily for 5 days to eyes of ocular normotensive nonhuman primates (cynomolgus monkeys), had no significant effects on total outflow facility and uveoscleral outflow compared to baseline and/or contralateral vehicle-treated control eyes. This indicates that the mechanism of the IOP lowering effects of abnormal cannabidiol may likely involve no or minor increases in aqueous outflow. Consequently, the intraocular pressure lowering effects of abnormal cannabidiol is anticipated to predominantly involve reduction of aqueous humor formation in humans and nonhuman primates.

It is apparent to one of ordinary skill in the art that different pharmaceutical compositions may be prepared and used with substantially the same results.

1. A method of treating glaucoma or ocular hypertension which comprises applying to the eye an amount sufficient to treat ocular hypertension of a combination of drugs which include a first drug which is a compound of formula I: 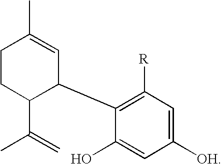
wherein R is selected from the group consisting of (CH₂)ₓ wherein x is 0 or an integer of from 1 to 7 and a second drug selected from the group consisting of cholinergic agonists, chlolinesterase inhibitors, prostamides and prostaglandins.

2. The method of claim 1 wherein said compound is a compound of formula

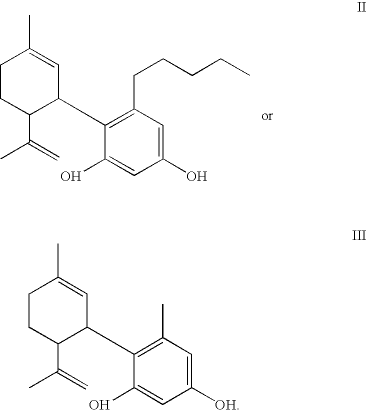

3. A pharmaceutical composition comprising a therapeutically effective amount of a combination drugs including a first drug which is a compound of formula I 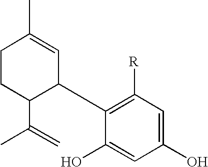
and a second drug selected from the group consisting of cholinergic agonists, chlolinesterase inhibitors, prostamides and prostaglandins.

4. The pharmaceutical composition of claim 3 which is an ophthalmic solution comprising a therapeutically effective amount of a combination of drugs including a first drug which is a compound of formula I 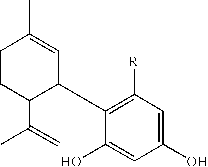
and a second drug selected from the group consisting of cholinergic agonists, chlolinesterase inhibitors, prostamides and prostaglandins.

5. The ophthalmic solution of claim 4 comprising at least one ingredient selected from the group of an ophthalmically acceptable preservative, buffer system, antioxidant and chelating agent.

6. The ophthalmic solution of claim 5 wherein said compound is of the formula

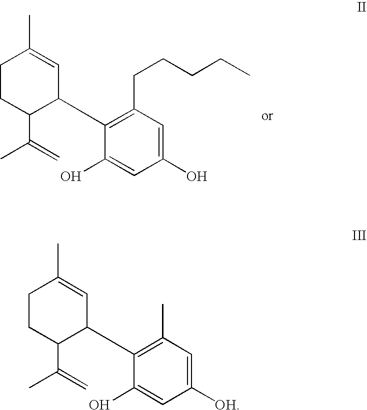

7. A pharmaceutical product, comprising
a container adapted to dispense its contents in metered form; and
an ophthalmic solution therein, as defined in claim 4.

8. A method for treating glaucoma or elevated intraocular pressure which comprises applying to the eye an amount sufficient to treat ocular hypertension of a combination of drugs which include a first drug which is a compound having Abnormal Cannabidiol activity and a second drug capable of increasing aqueous outflow of the eye.

9. The method of claim 1 wherein said second drug is a cholinergic agonist selected from the group consisting of charbachol, pilocarpine hydrochloride, pilocarpine nitrate, pilocarpine or pharmaceutically acceptable salts or prodrugs thereof

10. The method of claim 1 wherein said second drug is a cholin esterase inhibitor selected from the group consisting of demecarium, echothiophate, physostigmine, and the like, or pharmaceutically acceptable salts or prodrugs thereof.

11. The method of claim 1 wherein said second drug is bimatoprost or a pharmaceutically acceptable sale or prodrug thereof.

12. The method of claim 1 wherein said second drug is a prostaglandin selected from the group consisting of travoprost, UFO-21, cloprostenol, fluprostenol, 13,14-dihydro-cloprostenol, isopropyl unoprostone, latanoprost or pharmaceutically acceptable salts or prodrugs thereof.
